(19) United States
(12) Patent Application Publication
(10) Pub. No.: US 2002/0001584 A1
(43) Pub. Date: Jan. 3, 2002

(54) Tissue glue with improved antiadhesive properties

(75) Inventors: Hubert Metzner, Marburg (DE); Gerhard Dickneite, Marburg (DE); Monika Kroez, Lahntal (DE)

Correspondence Address: Finnegan, Henderson, Farabow, Garrett & Dunner, L.L.P., 1300 I Street, N.W., Washington, DC, 20005-3315, US

(73) Assignee: Aventis Behring GmbH

(21) Appl. No.: 09/861,657

(22) Filed: May 22, 2001

(30) Foreign Application Priority Data
May 22, 2000 (DE) 100 25 001.7

(51) Int. Cl. A61L 24/00; A61K 38/48

(52) U.S. Cl. 424/94.64

(57) ABSTRACT

Tissue glue with improved antiadhesive properties The use of a tissue glue which comprises a stabilized fibrinogen preparation which can be stored in the liquid or frozen state and comprises a chaotropic substance, and a thrombin preparation, for reducing or preventing postoperative tissue adhesions is described.

The invention relates to the use of a tissue glue for reducing or preventing postoperative tissue adhesions which is distinguished from known tissue glues by improved antiadhesive properties.

It is known that the development of tissue glues to date has given priority to the hemostatic activity or the sealing (e.g. against loss of CSF). These indications still account for a very considerable number of the uses of tissue glues nowadays.

Nevertheless, the preclinical or clinical use of tissue glues for avoiding adhesion after surgical intervention have also been described in the past—with varying success. Thus, H. Moro et al. reported inhibition of pericardial adhesions in a dog model (H. Moro, J. Hayashi, H. Ohzeki, T. Nakayama, O. Namura, K. Hanzawa and N. Yagi. Jap J Thor Cardiovasc Surg 47: 79-84, 1999). H. Takeuchi et al. and P. A. De Iaco et al. also describe the successful use of tissue glues for avoiding or reducing adhesions on the horn of the rabbit uterus (H. Takeuchi, Y. Toyonari, N. Mitsuhashi and Y. Kuwabara. J Obstet Gynaecol 23: 479-484, 1997; P. A. De laco, A. Costa, G. Mazzoleni, G. Pasquinelli, L. Bassein and A. Marabini. Fertil Steril 62: 400-404, 1994). The reduction in peritoneal adhesions has likewise been described by S. Lindenberg et al. on use of tissue glues in a rat model (S. Lindenberg and J. G. Lauritsen. Annales Chirurgiae and Gynecologiae 73: 11-13, 1984). However, there have also been other authors who observe no reduction in adhesions on use of fibrin glues by comparison with an untreated control. These were, inter alia, J. F. H. Gauwerki, J. Mann and G. Bastert, Arch Gynäkol Obstet 247: 161 (1990) and V. A. C. Evrard, A. De Bellis, W. Boeckx and I. A. Brosens, Hum Reprodt 11: 1877-1880 (1996). The reports, which are partly contradictory, are probably to be attributed essentially to the fact that the effect which can be achieved with the products available is insufficiently large or clear to lead consistently to unambiguous results.

Very recently, the possibility of using fibrin layers for avoiding adhesions has also been mentioned in the patent literature. The international patent application WO 96/22115 describes a sheet-like material consisting of crosslinked fibrin and employed for preventing adhesions but not itself having hemostatic properties. In another embodiment, this material is produced in situ by using it, after a first tissue glue with hemostatic activity, as second tissue glue layer without hemostatic properties. However, these methods are either impractical, because the fixing of such a fibrin film is difficult, or laborious because two tissue glues must be employed in order to achieve both hemostatic activity and antiadhesive properties.

In addition, a preparation of fibrin or fibrinogen and a biocompatible or biodegradable polymer which forms a viscous solution and has antiadhesive properties is disclosed in international patent application WO 92/22312.

The object therefore was to develop a tissue glue which, while having good hemostatic properties, shows improved results in reducing or preventing tissue adhesions and, moreover, does without addition of polymers which form viscous solutions and have antiadhesive properties.

Because of their great medical importance, in recent years considerable research effort has been directed at the further development and improvement of the known tissue glues. This has also involved particular attention being paid to the improvement of the storability of tissue glues. Thus, German patent applications DE-A-198 53033, DE-A-198 61 158 and DE-A 100 12 732 describe tissue glues and components thereof which are distinguished, inter alia, by particularly long storability in the liquid and/or frozen state. Detailed investigation of these novel tissue glues has now shown that they also have other advantageous properties which open up additional and valuable possible uses thereof.

This is because it has emerged that these novel tissue glues have considerably improved antiadhesive properties without this involving the need to accept losses of their hemostatic properties. The particular antiadhesive properties of the novel tissue glues are evident both on untreated wounds and on wounds treated with conventional tissue glues. It is particularly surprising in this connection that distinctly improved effects, by comparison with conventional tissue glues, in reducing or preventing tissue adhesions is also achieved when the aforementioned novel tissue glues are employed. These effects are observed both in a typical animal model for investigating the reduction in adhesions, such as a lengthwise incision wound on the horn of the rabbit uterus, and on hemostatic use [lacuna] a partial resection of the rabbit liver.

The invention therefore relates to the use of a tissue glue comprising

a stabilized fibrinogen preparation which can be stored in the liquid and/or frozen state and to which a chaotropic substance is added, and

a thrombin preparation

for reducing or preventing postoperative tissue adhesions.

It is moreover possible to add to the tissue glue in addition a preparation containing coagulation factor XIII if the latter is not present in sufficient quantity, so that it is used as 3-component glue. This is because fibrin crosslinking which is as complete as possible can enhance the antiadhesive effect of a fibrin glue by the fibrin matrix being, for example, less amenable to fibrinolytic degradation. However, it is also possible to admix coagulation factor XIII to the fibrinogen preparation from the outset, so that a 2-component glue is employed. In the case of a 3-component glue, the mixing ratio of the components fibrinogen, factor XIII and thrombin can be chosen in a suitable way in order to achieve good mechanical properties of the glue. Examples of suitable mixing ratios are about 1:1:1 to about 10:1:1 or 10:1:2 or, in general, x:y:z where x≧z≧y.

The tissue glue used according to the invention comprises a chaotropic substance in the fibrinogen preparation. Examples of chaotropic substances which have proved suitable are arginine, guanidine, citrulline, urea or their derivatives or mixtures thereof. They are generally added to the fibrinogen preparation in amounts of from 0.1 to 1.0 mol/l, preferably in amounts of less than 0.5 mol/l.

The properties of the aforementioned novel tissue glues are further advantageously influenced by addition of an antifibrinolytic. The antifibrinolytic used is, for example, aprotinin, s-aminocaproic acid (EACA), p-aminomethylbenzoic acid (PAMBA) or one of their physiologically tolerated salts or derivatives.

The fibrinogen preparation may additionally comprise as stabilizers

an inorganic salt or

one or more physiologically tolerated salts of organic carboxylic acids, in particular of citric acid or of lactic acid, or

one or more amino acids or

a mono- or disaccharide or

a sugar alcohol

or one of their mixtures.

A beneficial effect on the antiadhesive properties of the claimed, improved fibrin glues can further be achieved by suitable purification methods, for example by reducing the plasminogen content of the fibrinogen component. Examples of possible methods of this type are immunoaffinity chromatography via coupled antibodies or affinity chromatography on amino group-containing supports. This invention therefore also encompasses inter alia fibrin glues with fibrinogen components whose plasminogen contents have been significantly reduced. Such fibrinogen components preferably have a plasminogen to fibrinogen ratio of <1.8×10⁻⁴ (w/w), particularly preferably of <10⁻⁴ (w/w).

The factor XIII preparation added to the tissue glue to be employed according to the invention must likewise be stabilized if it is not added to the previously stabilized fibrinogen. In this case, it is advantageous to add to the factor XIII preparation a physiologically tolerated salt of an organic di-, tri- or tetracarboxylic acid, in particular of citric acid, and, where appropriate, other stabilizers and/or buffer substances for factor XIII. Other stabilizers suitable in this connection are

a mono- or disaccharide or a sugar alcohol and/or

an amino acid from the group of glycine, glycylglycine, alanine, cysteine, histidine, glutamine or a physiologically tolerated salt of glutamic or aspartic acid and/or

a reducing or oxidation-preventing agent and/or

a surface-active substance.

They are normally added in an amount of up to 5% by weight of the factor XIII preparation. Tissue glues of this type are described in German patent applications DE-A-198 53 033 and DE-A-198 61 158.

The thrombin preparation present in the tissue glue employed according to the invention displays in one embodiment the particular feature that it may contain a non-covalently binding inhibitor as stabilizer. Suitable substances for this purpose are compounds such as benzamidine or p-aminobenzamidine and other low to moderate affinity protease inhibitors. The addition of these low or moderate affinity inhibitors negligibly impairs the activity of thrombin in relation to substances such as fibrinogen. It is additionally possible to add to the thrombin preparation, besides a soluble calcium salt, for stabilization sodium chloride, a sugar or a sugar alcohol and/or an amino acid or else the salt of a mono- or polycarboxylic acid and/or the salt of a mono- or polyhydroxy carboxylic acid or mixtures of said stabilizers.

The thrombin used for this purpose is prepared from the prothrombin obtained from plasma or from a plasma fraction. After an activation thereof to thrombin without addition of thromboplastin and, where appropriate, further processing steps, it can be purified by a hydrophobic interaction chromatography and/or a cation exchange chromatography. This method is described in detail in German patent application DE-A-100 12 732.

It is particularly advantageous in this connection for the tissue glue or its constituents also to be subjected to one or more methods for inactivating viruses.

It is possible to use as starting material for producing the individual components of the fibrin glue of the invention apart from plasma fractions also recombinant proteins prepared by isolation from cell cultures or cell culture supernatants.

To investigate the effects of these improved tissue glues on the prevention or reduction of postoperative tissue adhesions, an improved tissue glue of the following composition was produced as an example:

Fibrinogen component containing:

90 mg/ml fibrinogen concentrate,

100 mmol/l NaCl,

20 mmol/l Na₃ citrate×2H₂O,

237 mmol/l L-arginine×HCl and

80 mmol/l ε-aminocaproic acid or 1000 KIU/ml aprotinin

Factor XIII component containing:

120 U/ml factor XIII concentrate,

10 mmol/l Na₃ citrate×2H₂O,

50 m/mol/l L-histidine

Thrombin component containing:

1 500 IU/ml thrombin concentrate,

150 mmol/l NaCl,

40 mmol/l CaCl₂,

110 mmol/l mannitol,

5 mmol/l L-histidine.

The pH after mixing the components to give the tissue glue was about 7.4.

The use of this tissue glue in operations is described by way of example below:

### EXAMPLE 1

### Prevention of Adhesions on the Horn of the Uterus

After opening the abdominal cavity of 12 female rabbits under anesthesia, longitudinal incisions were made in the horns of the uteri. The incisions were closed again with surgical suture material. Six rabbits were assigned to each of the two treatment groups as follows: 1. No treatment, 2. Treatment with improved tissue glue. The wounds in the second group were each completely covered with tissue glue. After closure of the abdominal cavity, the animals were allowed to return to consciousness. After euthanasia of the rabbits after 7 days, the adhesions of the uteri with the surrounding tissue were assessed. Adhesions of the two incisions together were excluded from the evaluation. The results of the investigation are shown in table 1.

The untreated animals showed adhesions in 63.6% of the cases. A distinct reduction in adhesions were observed in the group treated with improved tissue glue. The frequency of adhesions in this group was only 11.1%.

TABLE 1
Uterine adhesions with the surrounding tissue after
treatment with tissue glues

| | 1. No treatment | 2. Improved tissue glue |
|---|---|---|
| Frequency of | 63.6% | 11.1% |
| adhesions | | |

### EXAMPLE 2

### Prevention of Adhesions an the Horn of the Uterus

In this experiment, the improved tissue glue was compared with a commercial glue (Beriplast® P) and no treatment on a total of 36 rabbits. Three groups each of 12 animals were formed in accordance with the method described in example 1, and one horn of the uterus of each animal was treated as follows: 1. No treatment, 2. Beriplast® P, 3. Improved tissue glue. The frequency and extent of the adhesions were assessed on day 7. The results are summarized in table 2.

All the animals in the group which received no treatment with a tissue glue showed adhesions (100%). The rabbits treated with Beriplast® P had a distinctly lower frequency of adhesions (75%). The lowest frequency of adhesions was observed in the group of animals treated with improved tissue glue. The extent of the adhesions (length in cm) revealed similar findings.

TABLE 2
Adhesion of uterus with the surrounding tissue after
treatment with fibrin glues

| | | | 3. Improved |
|---|---|---|---|
| | 1. No treatment | 2. Beriplast ® P | tissue glue |
| Adhesion | 100% | 75% | 50% |
| frequency (%) | | | |
| Length of the | 1.52 | 1.03 | 0.67 |
| adhesions (cm) | | | |

### EXAMPLE 3

### Prevention of Adhesions on the Horn of the Uterus

In another experiment, improved tissue glues are compared with a commercial glue (Beriplast® P) and an untreated control. Several groups each of 12 animals were formed in accordance with the method described in example 2, using only one horn of the uterus of each animal. The animals were treated as follows:

1. No treatment

2. Beriplast® P

3. Improved tissue glue

4. Improved tissue glue (aprotinin in place of EACA)

5. Improved tissue glue with reduced plasminogen content

The frequency and the extent of the adhesions were assessed on day 7. Table 3 shows the results of the study.

About two-thirds of the animals which received no treatment with a tissue glue showed adhesions (66.7%). A distinctly lower frequency of adhesions or the lowest adhesion frequency was observed in the group of animals treated with improved tissue glues. The extent of the adhesions (length in cm) revealed similar findings.

TABLE 3
Adhesions of the uterus with the surrounding tissue after treatment
with various fibrin glues (mean for n = 12 animals)

| | | | | 4. Im- | |
|---|---|---|---|---|---|
| | | | | proved | 5. Im- |
| | | | | tissue | proved |
| | 1. Un- | | | glue | glue |
| | treat- | | 3. Im- | (apro- | with |
| | ed | 2. Beri- | proved | tinin in | reduced |
| | con- | plast ® | tissue | place of | plasminogen |
| | trol | P | glue | EACA) | content |
| Adhesion | 66.7% | 41.7% | 33.3% | 16.7% | 0% |
| frequency | | | | | |
| (%) | | | | | |
| Length of | 0.59 | 0.19 | 0.26 | 0.13 | 0 |
| the adhe- | | | | | |
| sions (cm) | | | | | |

### EXAMPLE 4

### Prevention of Adhesions on the Horn of the Uterus

In this experiment, several groups each of 8 animals was formed in accordance with the method described in example 1 and underwent operation on both horns of the uteri. Improved tissue glues were compared with a control group without treatment on 16 horns of the uteri in each group. The following treatment groups were compared:

1. No treatment

2. Improved tissue glue with reduced plasminogen content

3. Improved tissue glue with a plasminogen content which was initially reduced and was made up again before use

Only horns of the uterus which did not adhere to the incision in the other horn of the uterus were evaluated. The results of this series of tests (see table 4) show that the reduction in the plasminogen concentration can further improve the antiadhesive properties of a fibrin glue.

TABLE 4
Prevention of adhesions on the horn of the uterus by
treatment with fibrin glues (means)

| | | 2. Improved | 3. Improved tissue glue |
|---|---|---|---|
| | | tissue glue | after reducing the |
| | | with reduced | plasminogen content and |
| | 1. No | plasminogen | making up again with |
| | treatment | content | plasminogen |
| Adhesion | 68.8% | 15.4% | 46.2% |
| frequency (%) | | | |
| Length of the | 0.53 | 0.07 | 0.32 |
| adhesions (cm) | | | |

### EXAMPLE 5

### Prevention of Adhesions after Liver Resection

14 rabbits were anesthetized and, after opening of the abdominal cavity, the liver was exposed. A piece of about 3.5 g was resected from a lobe of the liver, resulting in a wound of about 4 cm². The wound was completely covered with a tissue glue to stop the bleeding, with 7 rabbits in each case receiving Beriplast® P or improved tissue glue. The number of animals in which the bleeding was completely stopped was recorded over 5 minutes. The abdominal cavity was then closed again and the anesthesia was terminated. After euthanasia of the animals after 7 days, the adhesions of the liver with the adjoining tissue were assessed.

Table 5 shows that the number of adhesions in the group treated with improved tissue glue was distinctly less than in the group treated with Beriplast® P. All the animals showed complete stoppage of the bleeding.

TABLE 5
Hemostasis and adhesion of the liver with the surrounding
tissue after treatment with tissue glues

| | 1. Beriplast ® P | 2. Improved tissue glue |
|---|---|---|
| Number of animals with | 5/7 | 2/7 |
| adhesions | (71.4%) | (28.6%) |
| Number of animals with | 7/7 | 7/7 |
| complete stoppage of | (100%) | (100%) |
| bleeding | | |

1. The use of a tissue glue comprising
a stabilized fibrinogen preparation which can be stored in the liquid and/or frozen state and to which a chaotropic substance is added, and
a thrombin preparation for reducing or preventing postoperative tissue adhesions.

2. The use of a tissue glue as claimed in claim 1, which additionally comprises a preparation which comprises coagulation factor XIII and which may also be mixed with the fibrinogen preparation.

3. The use of a tissue glue as claimed in claims 1 and 2, which comprises an antifibrinolytic.

4. The use of a tissue glue as claimed in claim 3, which comprises ε-aminocaproic acid as antifibrinolytic.

5. The use of a tissue glue as claimed in claim 3, which comprises p-aminomethylbenzoic acid as antifibrinolytic.

6. The use of a tissue glue as claimed in claim 3, which comprises aprotinin as antifibrinolytic.

7. The use of a tissue glue as claimed in claim 1-6, wherein the preparation of the fibrinogen component comprises additional purification steps.

8. The use of a tissue glue as claimed in claim 1-7, wherein the fibrinogen component has a reduced plasminogen content.

9. The use of a tissue glue as claimed in claim 8, wherein the ratio of plasminogen to fibrinogen is <1.8×10⁻⁴ (w/w).

10. The use of a tissue glue as claimed in claims 1 to 9, wherein
a physiologically tolerated salt of an organic di-, tri- or tetracarboxylic acid, in particular of citric acid, and
where appropriate other stabilizers and/or buffer substances for factor XIII
are added to the preparation containing factor XII.

11. The use of a tissue glue as claimed in claim 10, wherein
a mono- or disaccharide or a sugar alcohol and/or
an amino acid from the group of glycine, glycylglycine, alanine, cysteine, histidine, glutamine or a physiologically tolerated salt of glutamic or aspartic acid and/or
a reducing or oxidation-preventing agent and/or
a surface-active substance,
are added as other stabilizers to the preparation containing factor XIII.

12. The use of a tissue glue as claimed in claims 1 to 11, wherein one or more chaotropic substances from the group consisting of arginine, guanidine, citrulline, urea or their derivatives or mixtures thereof are added to the fibrinogen preparation.

13. The use of a tissue glue as claimed in claims 1 to 12, wherein additionally
an inorganic salt or
one or more physiologically tolerated salts of organic carboxylic acids, in particular of citric acid or of lactic acid, or
one or more amino acids or
a mono- or disaccharide or
a sugar alcohol
or one of their mixtures is added to the fibrinogen preparation as stabilizers.

14. The use of a tissue glue as claimed in claims 1 to 13, wherein a thrombin preparation which is stable in the liquid or frozen state and which, besides a soluble calcium salt and sodium chloride, may comprise as stabilizers
a buffer substance
a sugar or sugar alcohol and/or an amino acid and/or
a salt of a mono- or polycarboxylic acid or
a salt of a mono- or polyhydroxy carboxylic acid or mixtures of said stabilizers is employed.

15. The use of a tissue glue as claimed in claims 1 to 14, wherein the thrombin preparation comprises a non-covalently binding inhibitor as stabilizer.

16. The use of a tissue glue as claimed in claims 1 to 15, which comprises a thrombin preparation which is purified by a hydrophobic interaction chromatography, which may also be followed by a cation exchange chromatography.

17. The use of a tissue glue as claimed in claims 1 to 16, which has, or whose constituents have, been subjected to one or more methods for inactivating or removing viruses.

18. The use of a tissue glue comprising
a stabilized fibrinogen preparation which is storable in the liquid and/or frozen state and
a thrombin preparation, wherein the fibrinogen component has a reduced plasminogen content.
